(19) United States
(12) Patent Application Publication
(10) Pub. No.: US 2003/0125715 A1
(43) Pub. Date: Jul. 3, 2003

(54) Annuloplasty ring holder

(76) Inventors: Stephen T. Kuehn, Woodbury, MN (US); Thomas G. Schoon, Cedar, MN (US); Tory Martin, Prior Lake, MN (US); Bob Allan, Maple Groove, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 10/034,589

(22) Filed: Dec. 28, 2001

(51) Int. Cl. A61B 17/00; A61F 2/24

(52) U.S. Cl. 606/1; 623/2.11; 623/2.36

(57) ABSTRACT

An apparatus for holding an annuloplasty ring includes a holder body configured to hold the ring. A handle coupling attaches to a handle. A release mechanism on the handle coupling selectively releases the handle from the handle coupling.

### FIELD OF THE INVENTION

The present invention relates to the implantation of annuloplasty rings. More specifically, the invention relates to an annuloplasty ring holder system which aids in the implantation of the annuloplasty ring.

### BACKGROUND OF THE INVENTION

Certain types of diseases and defects in heart valves are known to reduce the efficiency of the natural valve. One such defect occurs when the annulus of the valve is enlarged or deformed such that the cusps of the natural valve do not form a seal when the valve is in a closed position. This allows regurgitation of blood through the cusps.

Such defects in the heart valve may be repaired using a surgical technique known as annuloplasty in which a prosthetic annuloplasty ring of varying shapes and varying compliances is affixed in the patient proximate the defective natural heart valve. Examples of annuloplasty rings are shown and described in French Patent No. 2 708 458 and U.S. Pat. No. 5,607,471.

During the implantation, an annuloplasty ring holder is used which releasably holds the annuloplasty ring. The annuloplasty ring holder is manipulated at the distal end of an elongated handle by the surgeon. The coupling between the distal tip of the handle and the ring holder should be such that the two may be easily separated. For example, it is desirable for the handle to be easily removed from the holder during implantation of the annuloplasty ring. At the same time, the annuloplasty holder should be securely attached to the handle to prevent unintentional separation of the holder from the distal end of the handle.

The prior art has used a number of techniques for coupling the distal end of the elongated handle to the holder. One such technique is the use of a tapered distal tip on the handle which is pressed into a similarly conforming tapered opening in the holder. This provides a friction fit between the holder and the distal tip of the handle which may be separated by applying a separation force between the two objects. This technique does not provide a positive lock between the handle and the ring holder. Therefore, it may be difficult to remove the handle from the holder when the holder is positioned adjacent to the natural valve because the separation force must be applied to the holder in the chest cavity while the handle is pulled from the holder. Alternatively, if the friction fit between the two pieces is too loose, the holder will slip off of the handle. It is difficult to accurately create the friction fit between the pieces because the size of the pieces may change due to minor dimensional variations. Thus, the engagement force may vary and could be difficult to control.

Another technique for coupling a handle to an annuloplasty ring holder is described in U.S. Pat. No. 5,290,300 to Cosgrove et al., entitled “FLEXIBLE SUTURE GUIDE AND HOLDER”. FIGS. 3 and 4 of the Cosgrove et al. reference show attaching a handle to a holder. The handle includes notch 48 which receives a spoke 39. The handle is coupled to the holder by forcing the two together and rotating the handle such that the spoke rests on a landing 50. The device described in the Cosgrove reference is undesirable because it presents a relatively large obstacle which makes viewing the native valve difficult.

Yet another example technique for coupling to an annuloplasty ring holder is illustrated in U.S. Pat. No. 5,843,177, entitled APPARATUS FOR ATTACHING A HANDLE TO AN ANNULOPLASTY RING IMPLANTATION DEVICE, issued Dec. 1, 1998 to Vanney et al.

### SUMMARY OF THE INVENTION

An apparatus for holding an annuloplasty ring includes a holder body configured to hold the annuloplasty ring. A holder body includes a handle coupling that couples to a handle. A release mechanism selectively releases the handle from the handle coupling.

### BRIEF DESCRIPTION OF THE DRAWINGS

FIG. 1A is an exploded perspective view of a holder apparatus in accordance with one embodiment of the present invention.

FIG. 1B is a side cross-sectional view of the holder of FIG. 1A.

FIG. 2 is a perspective view of a handle of the invention for use with the holder apparatus of FIG. 1.

FIG. 3 is a top plan view of the holder apparatus of FIG. 1.

FIG. 4 is a bottom plan view of the holder apparatus of FIG. 1.

FIG. 5 is a top perspective view of a holder apparatus in accordance with another embodiment.

### DETAILED DESCRIPTION OF THE PREFERRED EMBODIMENTS

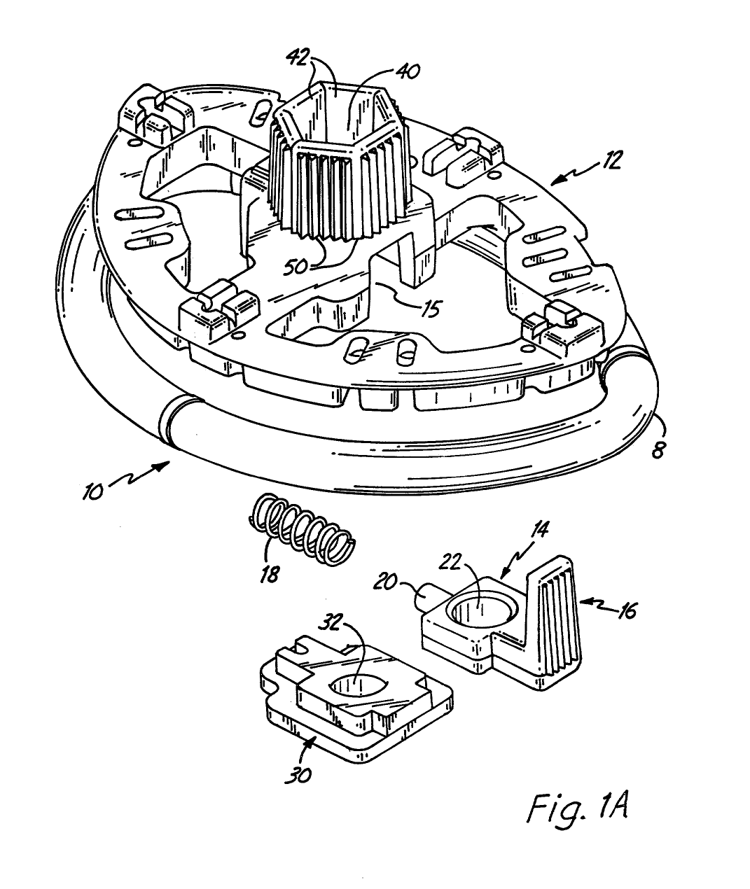

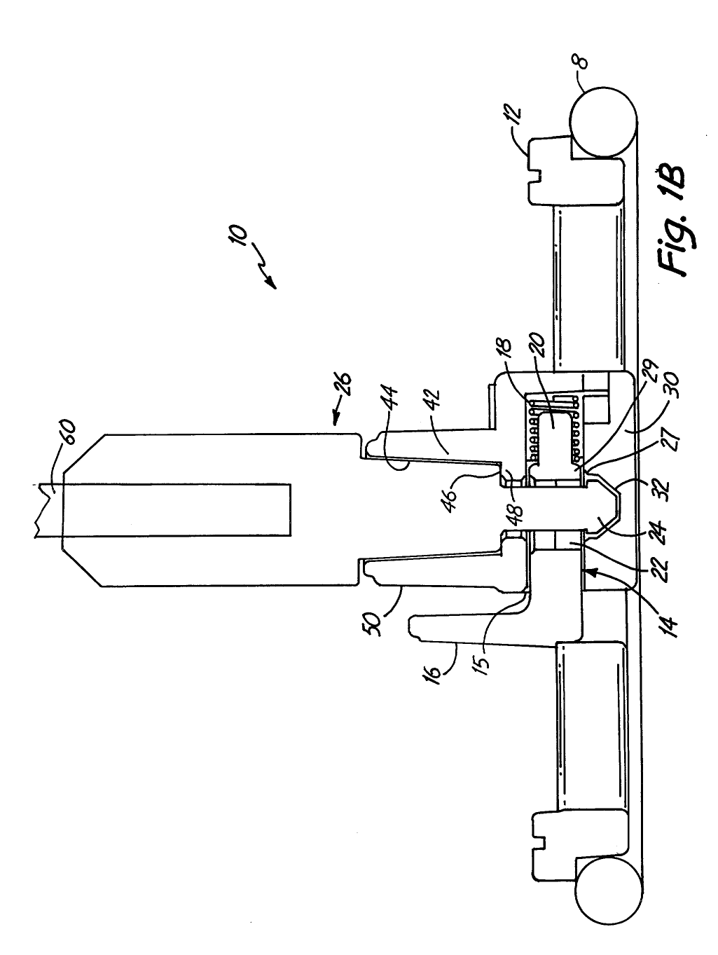

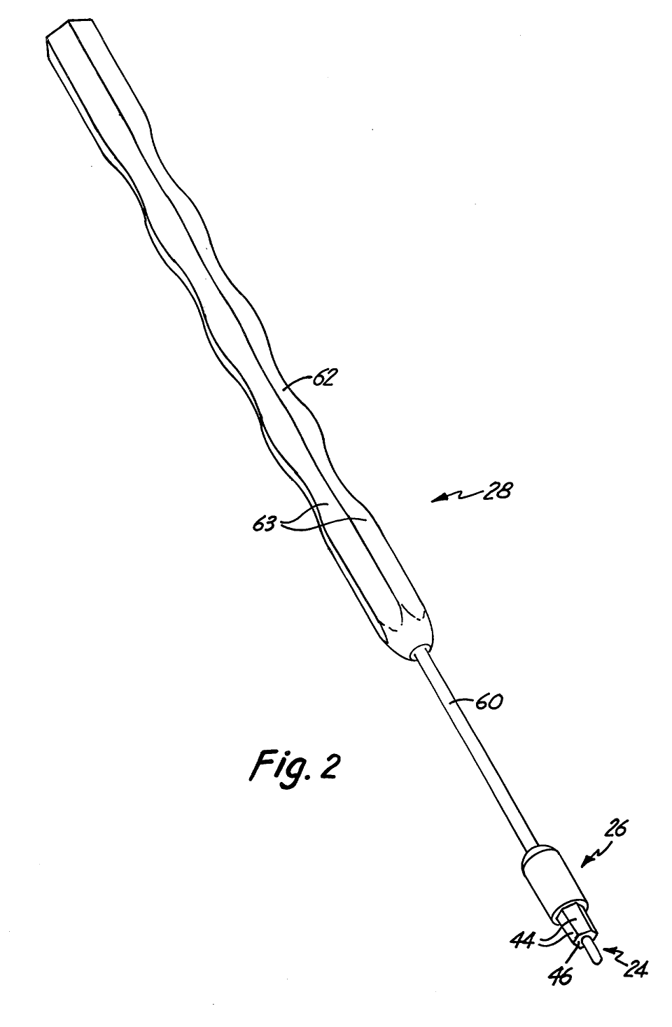

FIG. 1A is an exploded perspective view and FIG. 1B is a side cross-sectional view showing an annuloplasty ring holder apparatus 10 configured to hold an annuloplasty ring 8 during implantation. Holder apparatus 10 includes a holder body 12. Body 12 can have an opening for viewing or be solid. Annuloplasty ring 8 can be either a complete or partial ring and is attached to holder body 12 using any appropriate technique such as suturing, clamping and the like. The particular holder body configuration matches annuloplasty ring contours and the invention is not limited to the specific examples shown and discussed herein. The particular holder body configuration features shown herein are simply for illustration purposes and the invention is not limited to this configuration. A handle coupling 14 is slidably received in holder body 12 in cavity 15 and moves in the plane of body 12. A button 16 is configured to slidably actuate handle coupling 14 in a direction opposed to spring 18 which is seated on spring lock protrusion 20. The button 16 is an example of a release mechanism in accordance with the invention. An opening 22 in handle coupling 14 is configured to receive a knob 24 of tip 26 of a handle 28 shown in FIG. 2. Handle coupling 14 is secured in cavity 15 by back plate 30, for example, by welding, adhesive, snap fit and the like.

In operation, spring 18 acts as a bias force against the handle coupling 14, holding handle coupling 14 in a lock position in which knob 24 of tip 26 is locked in cavity 15 against the bottom of handle coupling 14. Tip 26 extends through opening 22 and knob 24 resides in, but does not touch, cup 32 of back plate 30. As shown in FIG. 1B, knob surface 27 abuts handle coupling surface 29. When button 16 is pressed, handle coupling 14 is moved to an unlock position within cavity 15, thereby releasing knob 24 of tip 26 such that handle 28 may be withdrawn from holder body 12.

Holder body 12 includes an opening 40 configured to receive tip 26. Opening 40 is shown including a number of flat side walls 42 which mate with sides 44 of tip 26. This configuration prevents rotation of tip 26 in holder body 12 so that the relative position of the holder to the handle remains constant. Additionally, tip 26 is tapered along sides 44 to match a taper of opening 40 to provide a more secure fit and provide automatic alignment of the tip 26 to opening 22. An abutting surface 46 of tip 26 abuts the bottom surface 48 of opening 40 to prevent the tip 26 from moving deeper into opening 40.

Surfaces 50 around opening 40 of body 12 are raised such that they can be easily gripped during surgery. During implantation, a surgeon can easily remove handle 28 from holder body 12 by pressing against button 16, thereby releasing knob 24. This allows the annuloplasty ring 8 to be sutured to the native tissue without interference from the handle or requiring the handle 28 to be secured to prevent excessive force from being applied onto body 12. Should the surgeon need to reinsert the handle 28, tip 26 is placed into opening 40 and knob 24 fits through opening 22 of handle coupling 14. This movement causes the angled tip of knob 24 to press against the side of opening 22, thereby moving or guiding handle coupling 14 into the unlock position. As tip 26 continues to be inserted, handle coupling 14 snaps back into the lock position once knob 24 has been completely inserted through opening 22. The insertion of the holder handle into the holder body is of low force.

In the perspective view of FIG. 2, handle 28 is shown as including a shaft 60 which couples a gripping portion 62 of handle 28 to tip 26. Shaft 60 can be a malleable section which allows the shaft to be bent to directly position the holder body 12 in front of the heart valve. In the particular embodiment, gripping portion 62 is ribbed to provide a low slip surface to be grasped by a surgeon. Flats 63 of gripping portion 62 are aligned with flat side walls 42 on holder 12, so that the ring can be aligned with anatomical features, such as the valve commissures and leaflets. Further, the flats may provide tactile feedback as to the ring orientation in relation to the commissures and the valve anatomy.

Preferably, the components of the holder apparatus 10 are comprised of biocompatible materials. Example materials include polyetherimide, polycarbonate, polysulfone, acetyl, polyetherether-ketone (PEEK), and metals such as titanium and stainless steel. Spring 18 can be of stainless steel, Nitinol (a nickel-titanium alloy), titanium or alloys thereof. These materials are provided as examples and the invention can be fabricated in other materials.

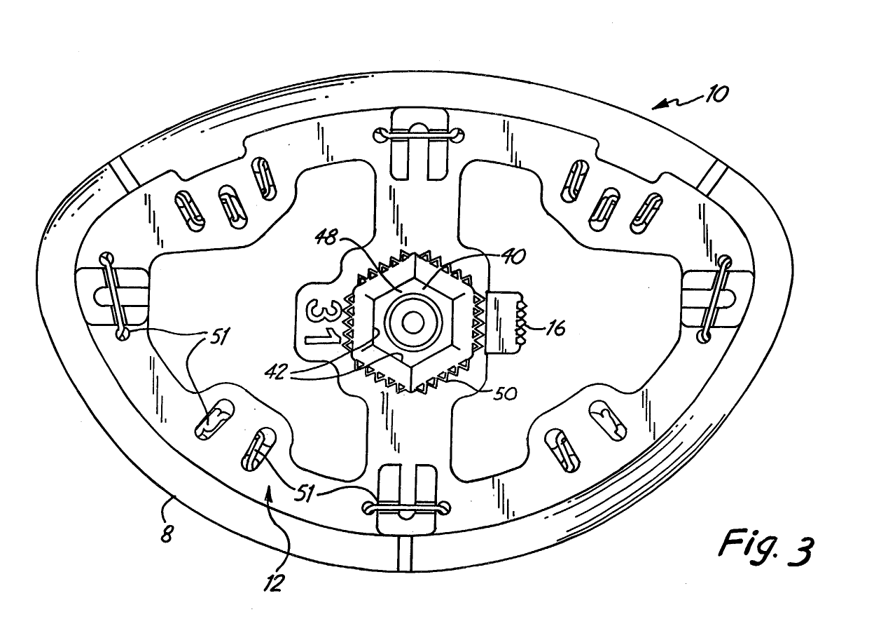

FIG. 3 is a top plan view of holder apparatus 10. The opening 40 and flat side walls 42 are clearly illustrated in FIG. 3. In this particular embodiment, there are six side walls 42 forming a hexagon, although it could be any other N sided and/or curved shape. The side walls 42 align and prevent rotation of the handle 28 in relation to the holder body 12. FIG. 3 also shows suture 51 which secures ring 8 to body 12.

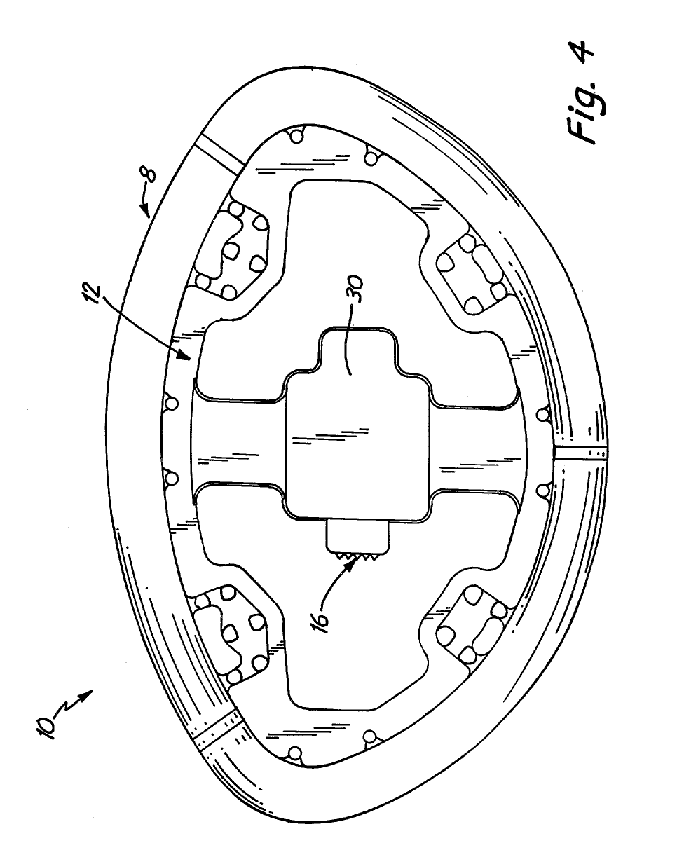

FIG. 4 is a bottom plan view of holder apparatus 10 and shows back plate 30 mounted onto holder body 12. The holder illustrated in FIGS. 1A, 1B, 3 and 4 uses suture(s) to couple the annuloplasty ring to holder body 12. The suture(s) are cut by the surgeon to release the ring. However, the invention can be used with any type of annuloplasty ring holder configured to couple to an annuloplasty ring using any technique.

The present invention provides a number of advantages. With the present invention, the handle can be easily inserted into and locked in the holder body. Further, as desired, the handle can be removed by actuating a button located on the holder body. The invention provides a position lock in both the axial and radial directions, between the handle and the holder body. Further, unintentional separation of the handle from the holder body is prevented. The holder can be re-inserted multiple times and allows insertion from only one surface (top). Further, the holder can be engaged from multiple radial orientations about the handle axis.

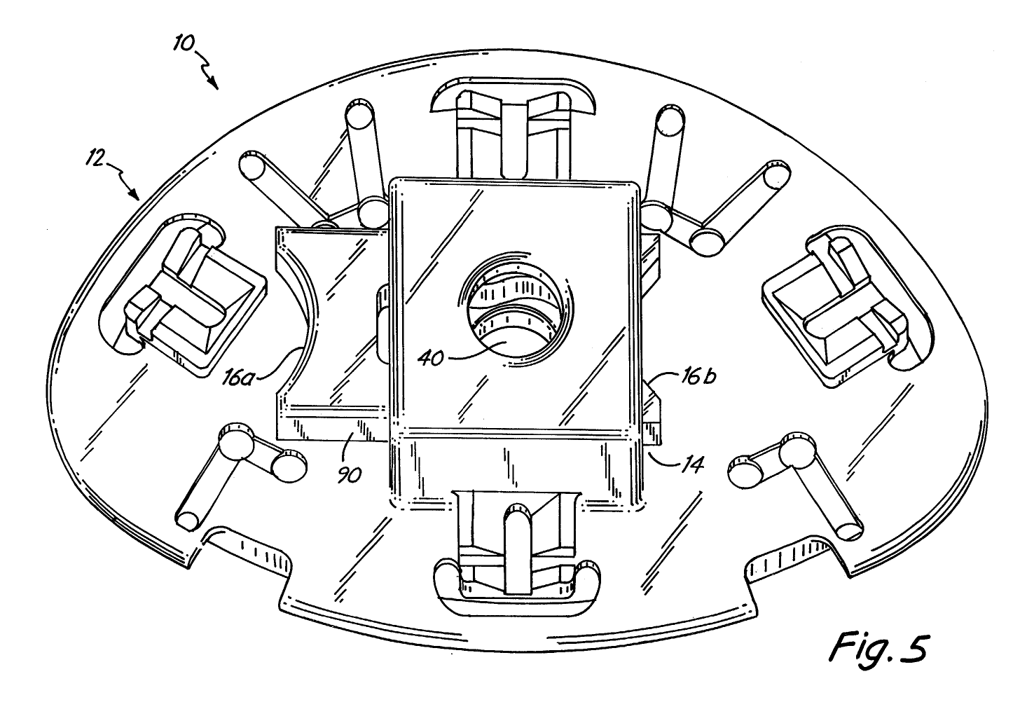

FIG. 5 is a top perspective view of another embodiment of annuloplasty ring holder apparatus 10. In FIG. 5, elements similar to those shown in FIGS. 1 and 3-4 are similarly numbered. FIG. 5 also shows another example of a holder body 12. In FIG. 5, clip 90 provides a handle coupling 14. Clip 90 slides in a plane parallel with the plane of holder body 12 and provides a locking and release mechanism. In the embodiment of FIG. 5, handle coupling 14 does not include a spring to move the handle coupling. Instead, the coupling 14 is pressed on surface 16a in one direction to lock handle coupling 14 onto the knob 24 of handle 28, and is pressed on surface 16b in the opposite direction to unlock knob 24 and release handle 28.

Although the present invention has been described with reference to preferred embodiments, workers skilled in the art will recognize that changes may be made in form and detail without departing from the spirit and scope of the invention. For example, other types of holder body configurations can be used. In one aspect, the particular handle coupling is not limited to the specific embodiment illustrated herein. The invention includes other types of handle couplings which can be actuated, or released, by pressing against a button carried on the holder body. Further, mechanisms other than a spring, and the particular spring illustrated, can be used to maintain the handle coupling by providing a bias force in a locked position. In one aspect, the handle coupling is configured to slide relative to a plane of the handle body.

What is claimed is:

1. An apparatus for holding an annuloplasty ring, comprising:
a holder body configured to the annuloplasty ring;
a handle coupling configured to couple the holder body to a handle; and
a release mechanism coupled to the handle coupling to release the handle from the handle coupling.

2. The apparatus of claim 1 including a spring configured to bias the handle coupling into a lock position.

3. The apparatus of claim 2 wherein the button is configured to transmit a force on the handle coupling against the spring toward an unlock position.

4. The apparatus of claim 1 wherein the handle coupling includes an opening formed therein to receive a tip of the handle.

5. The apparatus of claim 4 wherein the tip is locked within the opening.

6. The apparatus of claim 1 wherein the handle coupling is configured to couple to a knob at a tip of the handle.

7. The apparatus of claim 1 wherein the holder body includes an opening to receive the handle.

8. The apparatus of claim 7 wherein the opening is non-circular.

9. The apparatus of claim 7 wherein the opening is tapered.

10. The apparatus of claim 7 wherein a surface around the opening is raised to provide a gripping surface.

11. The apparatus of claim 1 wherein the handle coupling is configured to slide within the holder body in a direction generally parallel with a plane of the holder body.

12. The apparatus of claim 1 including a handle.

13. The apparatus of claim 12 wherein a tip of the handle includes a knob configured to engage the handle coupling.

14. The apparatus of claim 1 wherein the release mechanism comprises a button.

15. The apparatus of claim 1 wherein the handle coupling comprises a clip.

16. The apparatus of claim 15 wherein the clip is slidable within the holder body and provides a locking and release mechanism.
